(19) United States
(12) Patent Application Publication
(10) Pub. No.: US 2012/0290002 A1
(43) Pub. Date: Nov. 15, 2012

(54) TISSUE GRAFT ANCHORING

(75) Inventor: Steven William Astorino, Norfolk, MA (US)

(73) Assignee: Smith & Nephew, Inc., Memphis, TN (US)

(21) Appl. No.: 13/106,239

(22) Filed: May 12, 2011

(51) Int. Cl. A61B 17/04

(52) U.S. Cl. 606/232

(57) ABSTRACT

A method of securing a tissue graft includes providing a fixation member having a suture attached thereto, attaching the suture to the tissue graft, and adjusting the length of the suture between the fixation member and the tissue graft by pulling an end of the suture. A fixation device includes a member defining at least two openings and a suture passing through the at least two openings. The suture has a first end received within the suture through an open second end of the suture and exits the suture through a first opening in the suture between the first and second suture ends. A fixation device includes a suture having two closed loop portions that pass through openings in a member. A first suture end is received within the suture through a first opening in the suture, and a second suture end is received within the suture through a second opening in the suture.

This invention relates to tissue graft anchoring.

### BACKGROUND

An anterior cruciate ligament (ACL) that has ruptured and is non-repairable is generally replaced arthroscopically by a tissue graft. The tissue graft can be harvested from a portion of a patellar tendon having so called “bone blocks” at each end, and from the semitendonosis and gracilis. Alternatively, the tissue graft can be formed from synthetic materials or from a combination of synthetic and natural materials.

The replacement tissue graft is implanted by securing one end of the tissue graft in a socket formed in a passage within the femur, and passing the other end of the graft through a passage formed in the tibia. Generally, sutures are used to affix each end of the tissue graft to a fastener (e.g., an interference screw or a post), which is then secured to the bone.

It is also known to use a graft fixation member, e.g., a fixation button, to secure the tissue graft at the femoral cortex, as described in U.S. Pat. No. 5,306,301 (“the '301 patent”) hereby incorporated by reference in its entirety. When using a fixation button, the femoral passage generally includes a relatively larger diameter portion for receiving the graft, and a smaller diameter, passing channel near the femoral cortex for receiving a length of suture that runs from the tissue graft to the fixation button. By measuring the total length of the femoral passage and the length of the larger diameter portion of the femoral passage, the surgeon determines the appropriate length of suture material for attaching the fixation button to the tissue graft.

### SUMMARY

To increase the graft/channel interface in femoral fixation for cruciate repair the distance between a graft fixation member and the tendon construct is reduced. The ability to minimize this distance is generally limited by fixation member flipping constraints, which results in less tendon in the femoral channel, or leads to making compromises in the size of the graft fixation member to accommodate the tendon and the flipping of the graft fixation member.

According to one aspect, a method of securing a tissue graft includes providing a fixation member having a suture attached thereto, attaching the suture to the tissue graft, and adjusting the length of the suture between the fixation member and the tissue graft by pulling an end of the suture.

Embodiments of this aspect may include one or more of the following features.

The suture passes through at least two openings in the fixation member and has a first end received within the suture through an open second end of the suture to form a loop having only one free end.

The method includes passing the fixation member, suture, and attached tissue graft through a bone passage, followed by adjusting the length of the suture between the fixation member and the tissue graft.

According to another aspect, a fixation device includes a member defining at least two openings and a suture attached to the member. The suture passes through the at least two openings in the member and has a first end received within the suture through an open second end of the suture and exits the suture through a first opening in the suture between the first and second suture ends.

Embodiments of this aspect may include one or more of the following features.

The suture first end exits the suture through a second opening in the suture between the first suture end and the first opening, and is received within the suture through a third opening in the suture between the first suture end and the first opening.

A portion of the suture between the second opening and the third opening is located on a first side of the member, and another portion of the suture that passes through the at least two openings in the member is located on a second side of the member. The member defines, for example, four openings.

According to another aspect, a fixation device includes a member and a suture attached to the member. The suture has two closed loop portions that pass through openings in the member, a first suture end received within the suture through a first opening in the suture, and a second suture end received within the suture through a second opening in the suture.

Embodiments of this aspect may include one or more of the following features.

The first and second suture ends extend within the suture along first and second respective portions of the suture.

The first and second suture ends exit the suture between the first and second suture ends, for example, through a common third opening in the suture.

The openings in the member extend non-perpendicularly through the member.

According to another aspect, a fixation device includes a member defining at least two openings and a suture attached to the member that passes through the at least two openings in the member. A first suture end is received within the suture through a first opening in the suture and a second suture end is received within the suture through a second opening in the suture. The first and second suture ends exit the suture between the first and second suture ends.

Embodiments of this aspect may include one or more of the following features.

The first and second suture ends exit the suture through a common third opening in the suture

The first suture end received through the first opening forms a first suture loop, and the second suture end received through the second opening forms a second suture loop.

The member defines at least three, four or five openings through which the suture passes.

The at least two member openings extend non-perpendicularly through the member.

According to another aspect, a method of securing a tissue graft includes providing a fixation member having suture passing through at least two openings in the fixation member; attaching the suture to the tissue graft; and adjusting the length of the suture between the fixation member and the tissue graft by pulling the suture ends. First and second ends of the suture are received within the suture and exit the suture between the first and second suture ends

Embodiments of this aspect may include one or more of the following features.

The first and second ends of the suture exit the suture through a common opening in the suture.

The method includes passing the fixation member, suture, and attached tissue graft through a bone passage, followed by adjusting the length of the suture between the fixation member and the tissue graft.

The details of one or more implementations are set forth in the accompanying drawings and the description below. Other features, objects, and advantages of the disclosure will be apparent from the description and drawings, and from the claims.

### BRIEF DESCRIPTION OF THE DRAWINGS

FIGS. 1A and 1B are illustrations of a tissue graft secured within the knee by a graft fixation member.

FIG. 2 is another view of the knee joint of FIG. 1.

FIG. 3 is an illustration of a fixation device according to a first embodiment.

FIG. 4 is an illustration of an alternative embodiment of a fixation device.

FIG. 5 is an illustration of an additional alternative embodiment of a fixation device.

FIGS. 6A and 6B are illustrations of an additional alternative embodiment of a fixation device having a suture forming multiple suture loops.

FIG. 7A is an illustration of an additional alternative embodiment of a fixation device having a suture forming multiple suture loops.

FIG. 7B is a top vies of a graft fixation member of the fixation device of FIG. 7A.

FIGS. 8A and 8B are isometric and cross-sectional views of an alternative embodiment of a graft fixation member.

FIG. 9 is an illustration of an additional alternative embodiment of a fixation device having a suture forming multiple suture loops.

### DETAILED DESCRIPTION

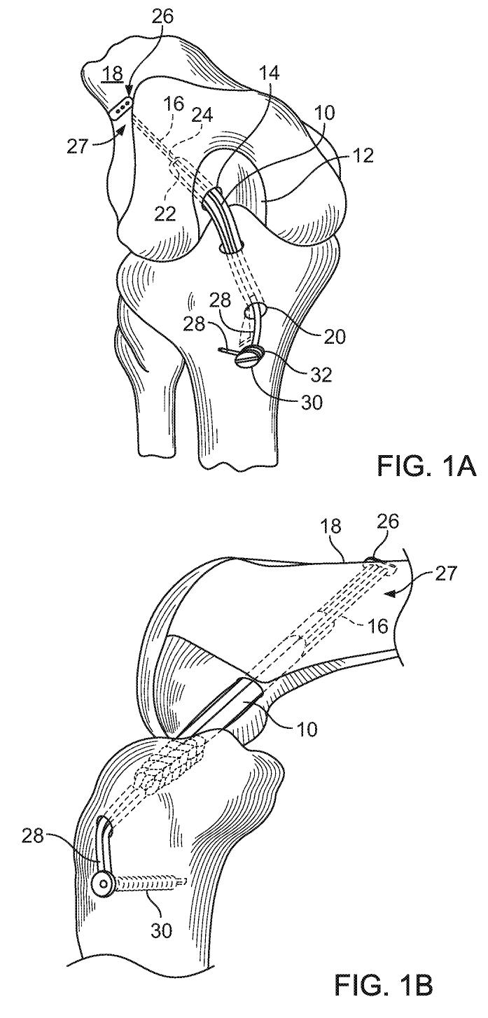

Referring to FIGS. 1A and 1B, a knee joint has a tissue graft 10 (e.g., a patellar tendon graft (FIG. 1A) or a semitendonosis and gracilis graft (FIG. 1B)) implanted in an anterior cruciate ligament (ACL) repair and reconstruction procedure. Prior to implanting tissue graft 10, a notchplasty procedure is preferably performed to expand the intercondylar notch 12 of the femur bone. A femoral channel 14 for receiving one end of tissue graft 10 is then drilled from notch 12 a predetermined distance within the femur with a passing channel 16 of reduced diameter drilled further through the femur from femoral channel 14 to a region of femoral cortex 18. A tibial channel 20 for receiving the other end of tissue graft 10 is drilled from an anterior region of the tibia to a region near the opening of femoral channel 14.

In the case of patellar tendon graft, one end of tissue graft 10 includes a bone block 22 which is shaped and sized in close conformity with femoral channel 14 to ensure optimal healing. A length of suture 24 has one end attached to bone block 22 and the other end secured at femoral cortex region 18 with a graft fixation member 26 of a fixation device 27. The suture 24 is attached to the graft fixation member 26 in a manner that permits the length of the suture 24 between the graft fixation member 26 and the tissue graft 10 to be adjusted prior to or after the graft 10 and the fixation member 26 have been positioned as shown in FIGS. 1A and 1B. The other end of tissue graft 10 includes a second length of suture 28 which is attached to the tibia, for example, with a fixation screw 30. A washer 32 either attached to or positioned under the head of fixation screw 30 helps in holding the suture in place when screw 30 is tightened.

The graft fixation member 26 is positioned using pull threads (not shown) attached to the member. The pull threads are passed through the channels 14 and 16 from the notch 12 to the cortex 18 and used to pull the graft fixation member 26 through the channels 14 and 16 with a long axis of the graft fixation member aligned with the channels. After exiting the channel 16, the pull threads are used to flip the graft fixation member 26 so that the member 26 lies flat against the cortex.

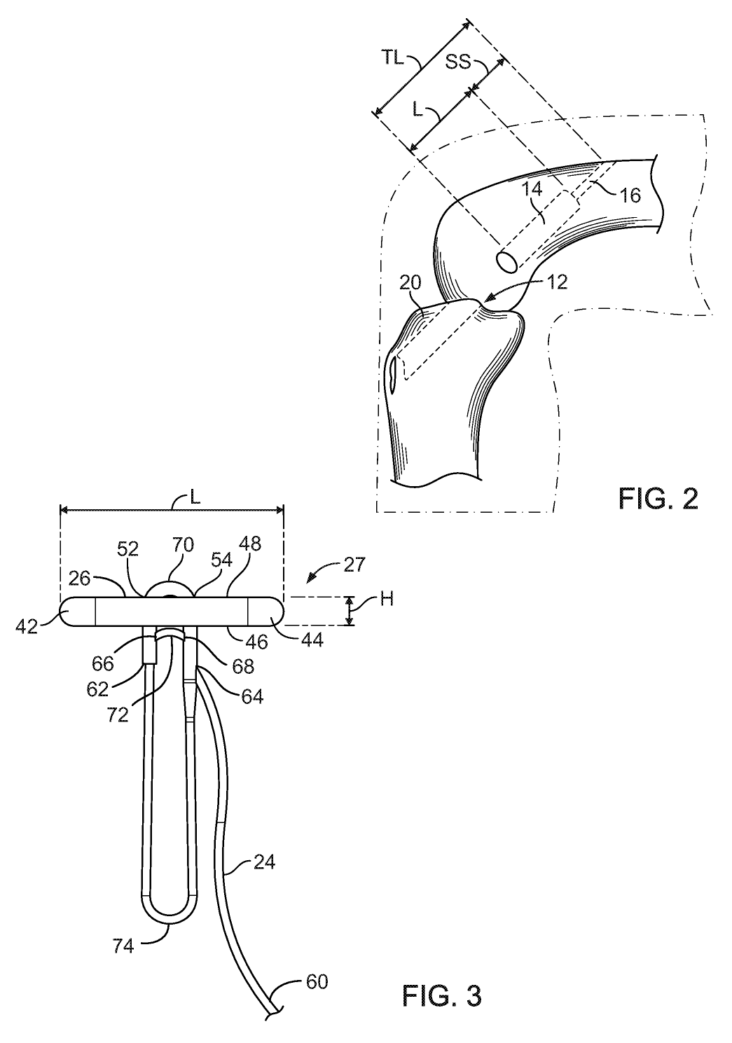

Referring to FIG. 2, length (L) of femoral channel 14 is selected by the surgeon in accordance with the length of bone block 22 and the desired insertion distance of tissue graft 10 within the femur. The span of suture 24, designated as SS, is approximately that of passing channel 16 so that the sum of the desired insertion length (L) and span of suture (SS) is the measured total length (TL) from the opening of femoral channel 14 to the opening at femoral cortex 18. Each of these dimensions is measured prior to implanting the tissue graft so that during the implantation procedure, the surgeon, under arthroscopic observation, can be assured that tissue graft 10 has been properly positioned within femoral channel 14.

The ability to adjust the length of the suture 24 allows the length of the suture span (SS) to be minimized, only being limited by the desired length of passing channel 16. There is a minimum length of the suture 24 that is necessary during passage of the graft fixation member 26 through the channel 14, 16 to allow the member 26 to be positioned in alignment with the channels. Once the graft fixation member 26 is located against the femoral cortex 18, the length of the suture 24 can be shortened by pulling on the suture to maximize the amount of the tissue graft 10 that is located within the femoral channel 14.

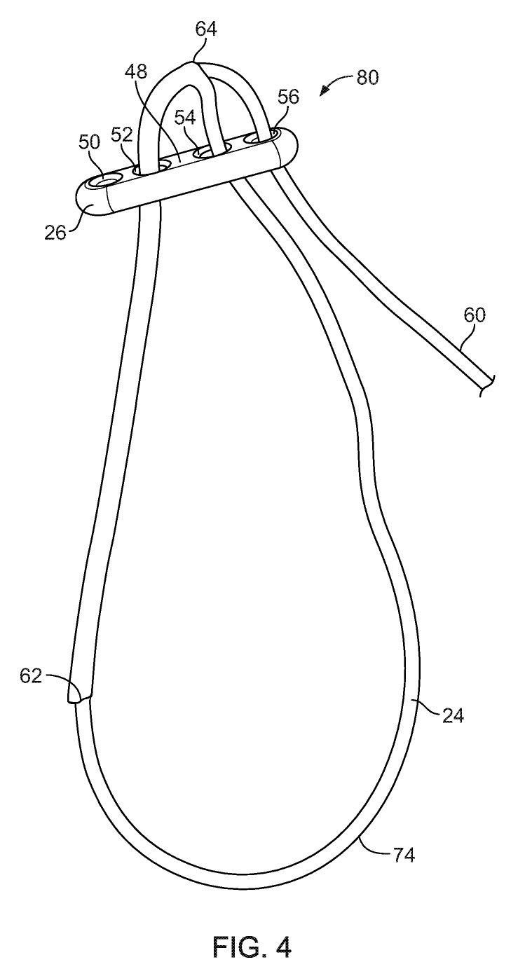

The suture 24 is a woven or braided suture, for example, #5 Ultrabraid suture, attached to the graft fixation member 26 in a loop configuration to form fixation device 27. Referring to FIG. 3A, the graft fixation member 26 is elongated in a first dimension defining a length, L, that extends between a first end 42 and a second end 44 of the member 26, has a second dimension transverse to the first dimension that has a width, W (not shown), smaller than the length, L, and has a third dimension transverse to the first and second dimensions that has a height, H, smaller than the length, L. The graft fixation member 26 has a bone contacting side 46 and an opposite side 48. Extending between the sides 46, 48 are at least two holes, for example, four holes 50, 52, 54 and 56 (see also FIG. 4). The holes 50 and 56 receive the passing sutures, not shown.

The suture 24 is a single length of suture attached to the graft fixation member 26. The suture 24 passes through openings 52, 54 in the graft fixation member 26, with a first end 60 of the suture received within the suture through an open second end 62 of the suture and exiting the suture through a first opening 64 in the suture between the first and second suture ends 60, 62. Between the end 62 and the first opening 64, the received suture exits the suture at a second opening 66 and reenters the sutures at a third opening 68. This suture configuration results in a portion 70 of the suture 24 that passes through the openings 52, 54 being positioned along the side 48 of the graft fixation member 26, a portion 72 of the suture 24 between the second opening 66 and the third opening 68 being positioned along the side 46 of the graft fixation member 26, and a suture loop 74 extending from the side 46 of the graft fixation member 26.

By pulling on the single free end 60 of the suture 24, the length of the loop 74 can be adjusted, for example, from a minimum that equals the distance between the holes 52 and 54 to about 30 mm, such that, with the tissue graft 10 attached the suture 24, the distance between the graft fixation member 26 and the tissue graft 10 can be adjusted between 0 mm and about 15 mm.

During manufacture, the suture end is threaded through a needle and the needle is passed through the center of the suture. A hot knife is used to melt bond the suture surrounding the openings to limit unraveling of the suture.

When the fixation device 27 is used with a semitendonosis and gracilis graft (FIG. 1B), the suture 24 can be provided to the surgeon pre-attached to the graft fixation member 26, and operating room personnel attach the tissue graft to the fixation device 27 by passing the tissue graft through the suture loop 74. When a patellar tendon graft (FIG. 1A) is used, operating room personnel form the loop configuration by passing the suture through the bone block of the graft while forming the loop configuration.

The distance between the graft fixation member 26 and the tissue graft 10 can be adjusted while the graft/fixation member construct is in the femoral tunnel. Alternatively, the distance is determined by the surgeon prior to placing the graft.

Once the graft fixation member 26 and the tissue graft 10 are positioned in the knee with the desired length of the suture 24, tension placed on the suture by the tissue graft acts to secure the loop configuration by the suture tightening around the sections of suture that pass within the suture.

The length, width and height of the fixation member 26, is for example, 12 mm×4 mm×1.5 mm.

Other embodiments are within the scope of the following claims.

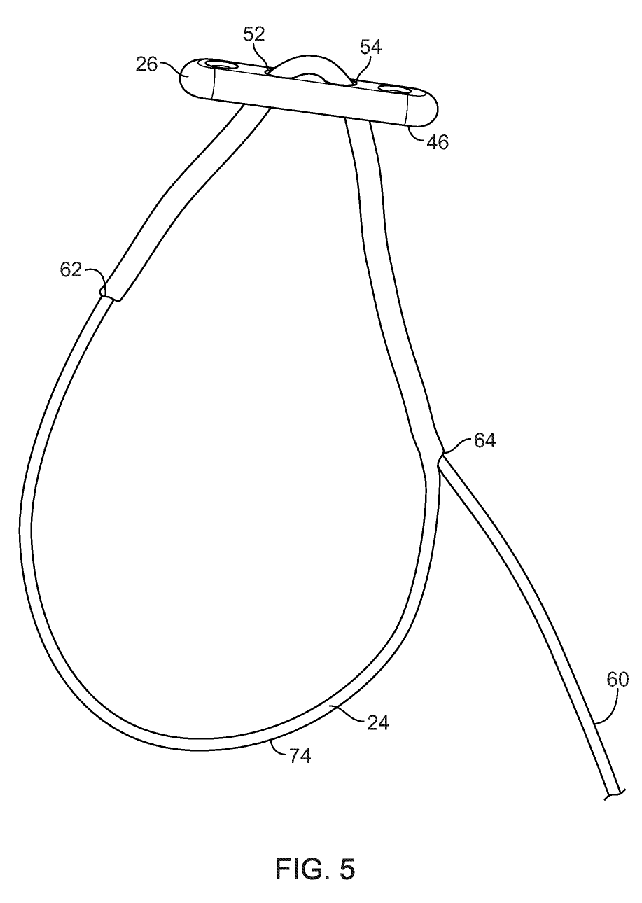

For example, referring to FIG. 4, rather than two entry and two exit openings in the suture 24, a fixation device 80 has the end 60 of the suture received through the open end 62 of the suture 24 and extending within the suture until reaching exit opening 64. The exit opening 64 is located on the side 48 of the graft fixation member 26, with the suture end 60 extending from the exit opening 64 through fixation member hole 56. Alternatively, as shown in FIG. 5, the exit opening 64 can be located on the side 46 of the graft fixation member 26.

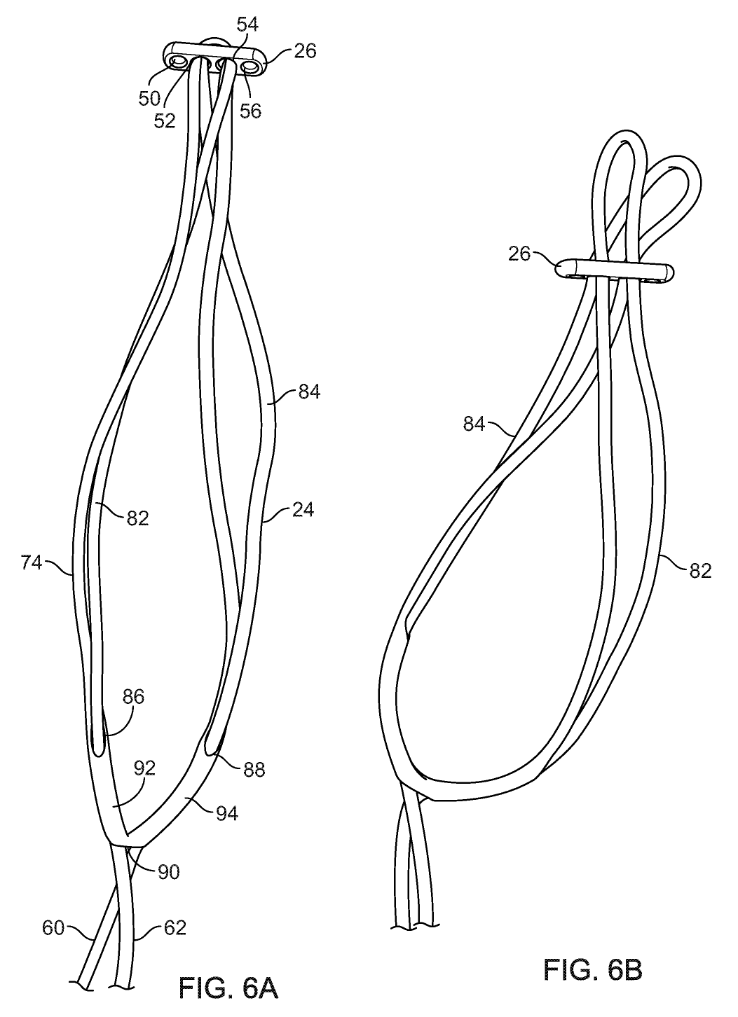

Referring to FIGS. 6A and 6B, the suture loop 74 can be formed by passing the ends 60, 62 of the suture through the openings 52, 54 in the graft fixation member 26 to form two separate, closed loop portions 82, 84 by passing the suture 24 through itself. The first end 60 of the single length of suture 24 is passed through opening 52, through opening 54 and then is received within a portion 92 of the looped suture through an opening 86 in the suture to form the closed loop portion 82. The second end 62 of the single length of suture 24 is passed through opening 54, through opening 52 and then is received within a portion 94 of the looped suture through an opening 88 in the suture to form the closed loop portion 84. The suture ends 60, 62 extend within the suture 24 along the first and second respective portions 92, 94 of the suture 24 and exit the suture 24 between the suture ends 60, 62, for example, through a common opening 90.

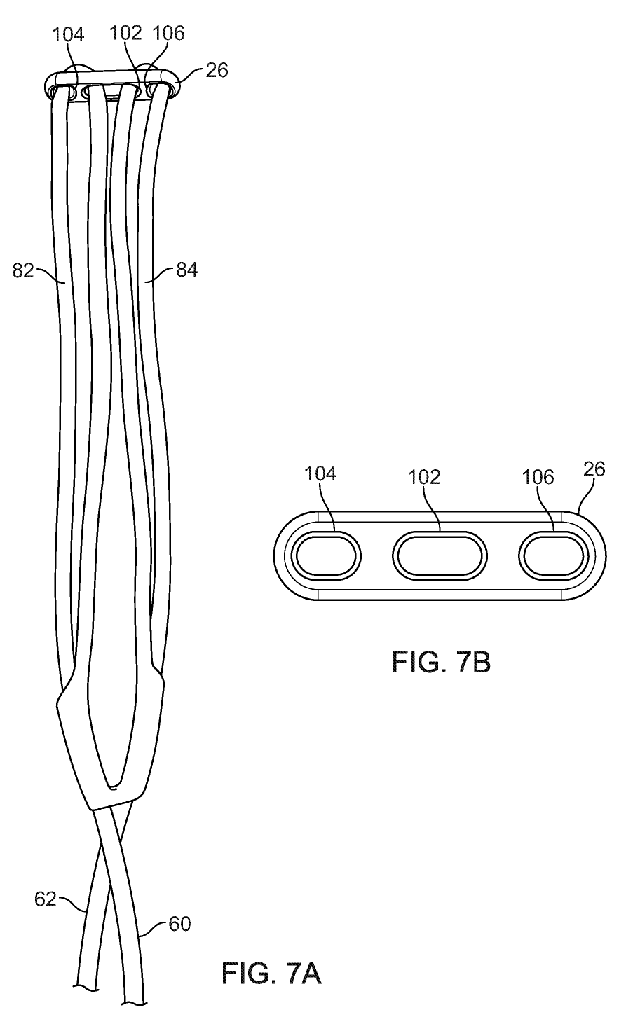

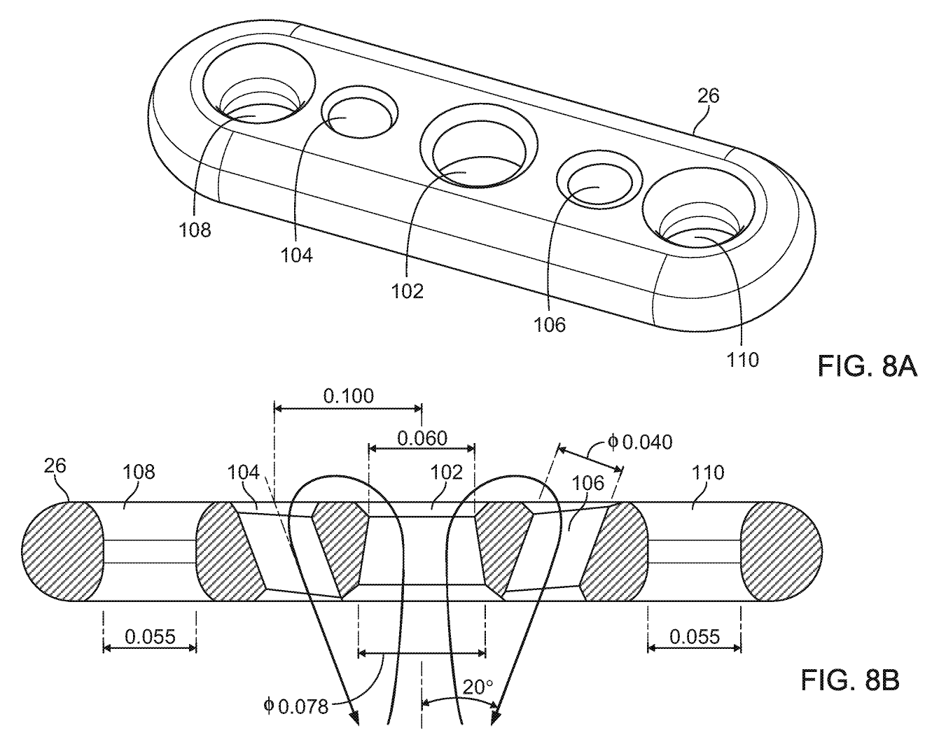

Rather than having the suture 24 form the suture loops 82, 84 by passing through the same openings 52, 54, as illustrated in FIGS. 7A and 7B, the suture ends 60, 62 can pass through a common opening 102 in the graft fixation device 26, with the end 60 then passing through an opening 104 in the graft fixation device 26, and the end 62 passing through an opening 106 in the graft fixation device 26. Referring to FIGS. 8A and 8B, rather than the three holes illustrated in FIG. 7B, the graft fixation device 26 can include two additional holes 108 and 110 that provide separate holes for the pull sutures. In the illustrated embodiment, openings 104 and 106 extend non-perpendicularly through the member, for example, at an angle of 20 degrees off perpendicular, creating a tortuous path for the suture to slide easier in one direction than the opposite direction.

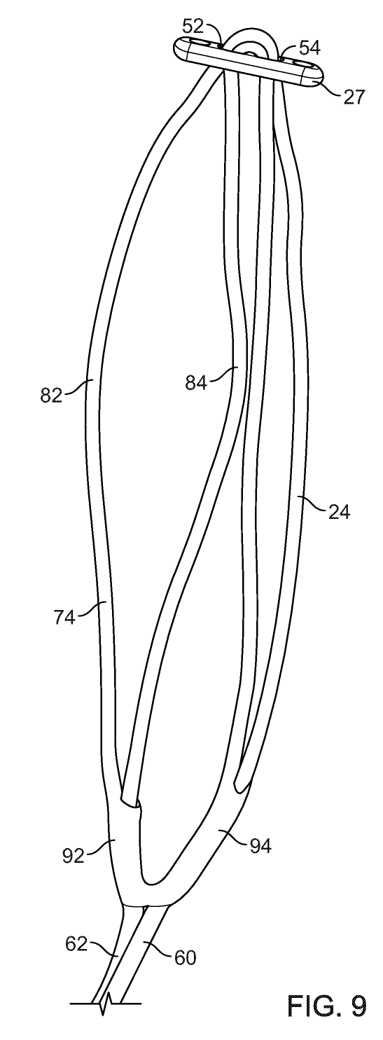

Rather than forming closed loop portions 82, 84 as described with respect to FIG. 6A, the closed loop portion 82 of FIG. 9 is formed by passing the suture end 60 through openings 52 and 54 and then through the second suture portion 94, and the closed loop portion 84 is formed by passing the suture end 62 through openings 54 and 52 and then through the first suture portion 92.

Other implementations are within the scope of the following claims.

For use in other applications, such as in AC joint repair or in other areas of the body to bring bone to bone, a second fixation member 26 can be coupled to the suture 24. For example, in any of the embodiments disclosed herein, the suture 24 can be threading through holes in the second fixation member. In use, the two fixation members 26 are located on opposing sides of the bones to be brought together, and the suture end(s) are pulled to bring bone to bone.

Furthermore, rather than using fixation member(s) that are rigid or formed of a material other than suture, the fixations member(s) can be formed of suture, such as described in U.S. patent application Ser. No. 13/016,3892, filed Jan. 28, 2011, hereby incorporated by reference herein in its entirety.

What is claimed is:

1. A method of securing a tissue graft comprising:
providing a fixation member having a suture attached thereto, the suture passing through at least two openings in the fixation member, the suture having a first end received within the suture through an open second end of the suture to form a loop having only one free end;
attaching the suture to the tissue graft; and
adjusting the length of the suture between the fixation member and the tissue graft by pulling the free end of the suture.

2. The method of claim 1 comprising passing the fixation member, suture, and attached tissue graft through a bone passage, followed by adjusting the length of the suture between the fixation member and the tissue graft.

3. A fixation device comprising:
a member defining at least two openings; and
a suture attached to the member, the suture passing through the at least two openings in the member, the suture having a first end received within the suture through an open second end of the suture and exits the suture through a first opening in the suture between the first and second suture ends.

4. The fixation device of claim 3 wherein the suture first end exits the suture through a second opening in the suture between the first suture end and the first opening, and is received within the suture through a third opening in the suture between the first suture end and the first opening.

5. The fixation device of claim 4 wherein a portion of the suture between the second opening and the third opening is located on a first side of the member, and another portion of the suture that passes through the at least two openings in the member is located on a second side of the member.

6. The fixation device of claim 3 wherein the member defines four openings.

7. A fixation device comprising:
a member; and
a suture attached to the member, the suture having two closed loop portions that pass through openings in the member, the suture having first and second ends, the first suture end received within the suture through a first opening in the suture and the second suture end received within the suture through a second opening in the suture, the first and second suture ends extending within the suture along first and second respective portions of the suture and exiting the suture between the first and second suture ends.

8. The fixation device of claim 7 wherein the first and second suture ends exit the suture through a common third opening in the suture.

9. The fixation device of claim 7 wherein the openings in the member extend non-perpendicularly through the member.

10. A fixation device comprising:
a member defining at least two openings; and
a suture attached to the member, the suture passing through the at least two openings in the member, the suture having first and second ends, the first suture end received within the suture through a first opening in the suture and the second suture end received within the suture through a second opening in the suture, the first and second suture ends exit the suture through a common third opening in the suture between the first and second suture ends.

11. The fixation device of claim 10 wherein the first suture end received through the first opening forms a first suture loop, and the second suture end received through the second opening forms a second suture loop.

12. The fixation device of claim 10 wherein the member defines at least three openings through which the suture passes.

13. The fixation device of claim 10 wherein the member defines at least four openings.

14. The fixation device of claim 10 wherein the member defines five openings.

15. The fixation device of claim 10 wherein the at least two member openings extend non-perpendicularly through the member.

16. A method of securing a tissue graft comprising:
providing a fixation member having a suture attached thereto, the suture passing through at least two openings in the fixation member, the suture having first and second ends which are received within the suture and exit the suture through a common opening in the suture between the first and second suture ends;
attaching the suture to the tissue graft; and
adjusting the length of the suture between the fixation member and the tissue graft by pulling the suture ends.

17. The method of claim 16 comprising passing the fixation member, suture, and attached tissue graft through a bone passage, followed by adjusting the length of the suture between the fixation member and the tissue graft.
